(19) United States
(12) Patent Application Publication
(10) Pub. No.: US 2024/0315545 A1
(43) Pub. Date: Sep. 26, 2024

(54) Tongue Depressor and Illumination Support Device for Oral and Throat Imaging

(71) Applicants: Haluk Un, WOODINVILLE, WA (US); MUSTAFA YASAR SIR, WOODINVILLE, WA (US)

(72) Inventors: Haluk Un, WOODINVILLE, WA (US); MUSTAFA YASAR SIR, WOODINVILLE, WA (US)

(21) Appl. No.: 18/609,051

(22) Filed: Mar. 19, 2024

Related U.S. Application Data
(60) Provisional application No. 63/453,297, filed on Mar. 20, 2023.

(51) Int. Cl. A61B 1/24; A61B 1/00; A61B 1/04; A61B 13/00

(52) U.S. Cl. CPC A61B 1/24; A61B 1/000095; A61B 1/0014; A61B 1/042; A61B 13/00

(57) ABSTRACT

The invention disclosed herein pertains to a support device specifically designed to aid in the imaging of the oral and throat regions. This device is characterized by the integration of a tongue depressor and a supporting body. The tongue depressor possesses a flat body with a rounded distal end and an opposite proximal end. The supporting body is distinguished by a rear surface that is capable of detachable coupling with an image capture device, alongside an opposing front surface designed to securely attach to the proximal end of the tongue depressor. The configuration ensures that the tongue depressor is maintained in an orientation perpendicular to the image plane of any attached image capture device.

### FIELD OF INVENTION

The present invention relates generally to the field of medical imaging devices, specifically to an apparatus designed to facilitate the imaging of the oral and throat regions.

### BACKGROUND

The endeavor to accurately image the oral and throat regions presents a series of substantial challenges that have persisted despite advances in medical and imaging technologies. The primary difficulties arise from the intrinsic conditions within the oral cavity, notably its inherent darkness and the obstruction caused by the tongue, which severely limits the visibility of the throat area. These obstacles necessitate the use of auxiliary tools, such as tongue depressors and illumination devices, to facilitate a clear view during examinations or imaging procedures.

In traditional practices, healthcare professionals have had to manage these tools concurrently, a process that not only requires significant skill but also complicates the procedure, potentially compromising patient comfort and the efficiency of the examination. The integration of illuminating capabilities with a tongue depressor has been proposed as a solution, aiming to streamline the process by consolidating these essential functions into a single device. However, such devices often operate independently of the imaging equipment, necessitating their own power sources and, consequently, increasing both production costs and physical bulk. This not only limits their accessibility and convenience but also poses significant logistical challenges in terms of transportation and adaptability across different electronic imaging devices.

The advent of telemedicine and remote consultations has amplified the need for versatile and efficient imaging solutions that can be easily utilized with mobile electronic devices. Existing auxiliary devices, designed to aid in capturing clear images of the throat and oral cavity during video consultations, vary in their independence and specificity to certain electronic devices. This variability introduces a new set of limitations, including high energy requirements, elevated production costs, and a lack of universal compatibility, which restricts their widespread adoption and utility across different models and types of electronic devices.

The landscape of telemedical throat examination and orifice inspection systems, as exemplified by patents such as US20170014024A1, US 20220192468A1, JP2017119026A, and CN104323757A, reveals a pattern of complexity and high production costs that impede widespread adoption and user-friendly operation. The Telemedical throat examination device detailed in US20170014024A1 is characterized by its intricate design and operational mechanisms, leading to significant manufacturing expenses and challenges in user engagement. Similarly, the Orifice inspection system under US20220192468A1 requires an independent energy source for illumination, adding to the device's overall cost and complicating its manufacture. The Larynx camera unit and larynx camera set described in JP2017119026A incorporate a specialized camera and lighting system tailored for the throat and larynx areas. This specialization, while potentially beneficial for specific applications, contributes to the device's complexity and elevates production costs. Additionally, the Endoscope with flash lamp and camera for portable intelligent equipment, as outlined in CN104323757A, employs fiber optic connections for transmitting images and light, further exemplifying the trend towards sophisticated, high-cost devices within this field. These examples underscore the need for an innovative solution that simplifies the design and operation of throat and oral cavity imaging devices, thereby reducing production costs and enhancing usability for a broader range of applications.

Addressing these multifaceted challenges requires a novel approach that not only simplifies the imaging process but also enhances compatibility, reduces dependency on external power sources, and minimizes production costs. The ideal solution would seamlessly integrate with a variety of electronic devices, leveraging their existing capabilities to illuminate the oral cavity effectively without imposing additional burdens in terms of cost, space, or device specificity. This background underscores the pressing need for an innovative design that overcomes the limitations of current systems, thereby facilitating more efficient, cost-effective, and accessible imaging of the oral and throat regions.

It is within this context that the present invention is provided.

### SUMMARY

The present invention relates to a support device designed to assist in the imaging of the oral and throat regions. This device incorporates a tongue depressor with a flat body, featuring a rounded distal end and an opposite proximal end. A supporting body is included within the device, equipped with a rear surface for detachable coupling to an image capture device and an opposing front surface for secure attachment to the tongue depressor. The architecture ensures that the tongue depressor maintains a perpendicular orientation relative to the image plane of the image capture device when assembled, thus facilitating improved imaging of the targeted areas.

In some embodiments, the tongue depressor is constructed from materials such as wood, plastic, silicone, metal, glass, or combinations thereof, offering versatility and adaptability to various medical and environmental requirements while ensuring patient safety and comfort.

In another embodiment, the supporting body's rear surface features an adhesive material covered by a protective layer. When this layer is removed, the exposed adhesive surface enables easy and secure attachment to an image capture device, providing a stable imaging setup.

Some embodiments include suction cups on the supporting body's rear surface, allowing for detachable coupling to the image capture device. This feature adds an extra layer of convenience and adaptability, facilitating quick setup and removal without leaving residues.

In certain embodiments, the tongue depressor and supporting body are designed to attach to a case, which can then attach to the image capture device. This configuration ensures compatibility with a wide range of mobile electronic devices, enhancing the utility and applicability of the device in various medical settings.

In some embodiments, the tongue depressor includes a reflective layer on at least a portion of its surface. This innovative feature enhances illumination within the oral cavity, improving visibility and image quality without the need for additional light sources.

Another embodiment involves the supporting body being made from at least partially transparent material. This design allows the supporting body to conduct light from the image capture device's light source to the oral cavity, illuminating the area naturally and effectively.

In some embodiments, the supporting body includes a light-reflecting layer, which works in conjunction with the partially transparent material to direct light towards the oral and throat regions, further enhancing illumination and image clarity.

Some embodiments feature a front surface of the supporting body that couples to the tongue depressor via a recessed area with grooves. This configuration securely holds the tongue depressor and allows for the adjustment of its orientation, enabling optimal imaging angles and versatility in usage.

An embodiment includes an optical cover with light-transmitting properties connected to the supporting body. This cover is designed to align with the camera and light source of the image capture device, improving image capture by directing light efficiently towards the targeted area without interfering with the camera's field of view.

In some embodiments, the device utilizes artificial intelligence models to optimize image capture parameters such as stability, lighting, distance, and zoom and cropping settings. This technological integration enhances the quality of images collected, providing support for medical diagnostics and patient consultations.

Furthermore, some embodiments incorporate classifiers for image-quality assessment and deep learning models for assisting in the optimal positioning of the device. Additionally, pixel-level image fusion models are used to produce the final image, showcasing the device's capability to leverage advanced technologies for improved medical imaging outcomes.

### BRIEF DESCRIPTION OF THE DRAWINGS

Various embodiments of the invention are disclosed in the following detailed description and accompanying drawings.

FIG. 1A is a top view of an example configuration of the tongue depressor.

FIG. 1B is a perspective view of an example configuration of the tongue depressor.

FIG. 1C is a front view of an example configuration of the holder body.

FIG. 1D is a side cross-sectional view of an example configuration of the holder body.

FIG. 2A is a front-perspective view of an example configuration of the holder body.

FIG. 2B is a back-perspective view of an example configuration of the holder body.

FIG. 3A is a back-perspective view of an example configuration of the holder body.

FIG. 3B is a perspective view of an example configuration of the tongue depressor and holder body.

FIG. 4A is a perspective view of an example configuration of the tongue depressor and holder body.

FIG. 4B is a perspective view from the front of an example configuration of the tongue depressor, holder body, and electronic device case.

FIG. 5 is a perspective view from the back of an example configuration of the tongue depressor, holder body, and electronic device case.

FIG. 6A is a perspective view of an example configuration of the tongue depressor and holder body attached to an electronic device.

FIG. 6B is a perspective view from the front of an example configuration of the tongue depressor, holder body, and electronic device case attached to an electronic device.

FIGS. 7A and 7B are side views of an example configuration of the holder body.

FIG. 7C is a top view of an example configuration of the holder body.

FIG. 7D is a side cross-sectional view of an example configuration of the holder body.

FIG. 8A is a perspective view of an example configuration of the holder body.

FIG. 8B is a perspective view of an example configuration of the holder body attached to an electronic device.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### Detailed Description and Preferred Embodiment

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terms “about” and “approximately” indicate an acceptable degree of error or variation in measurements, usually within 20%, preferably within 10%, and more preferably within 5% of a given value or range. Numerical values provided in this description are approximate unless stated otherwise.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

The use of spatial terms, such as “under,” “below,” “lower,” “over,” “upper,” etc., is used for ease of explanation to describe the relationship between elements when the apparatus is in its proper orientation.

The terms “first,” “second,” and the like are used to distinguish different elements or features, but these elements or features should not be limited by these terms. A first element or feature described can be referred to as a second element or feature and vice versa without departing from the teachings of the present disclosure.

As used herein, the term ‘support device’ encompasses any apparatus or assembly designed to assist in the imaging of the oral and throat regions by holding or positioning a tongue depressor in relation to an image capture device. The support device may be constructed from various materials including, but not limited to, plastics, metals, silicone, or combinations thereof, and may be manufactured using processes such as injection molding, 3D printing, or any other suitable manufacturing technique.

The term ‘image capture device’ as used herein refers to any electronic device equipped with imaging capabilities, including, but not limited to, digital cameras, smartphones, tablets, and specialized medical imaging equipment. The image capture device may possess functionalities such as video recording, still photography, and live streaming, and may include additional features such as built-in illumination sources (e.g., LED lights) to enhance image quality.

For the purposes of this disclosure, a ‘tongue depressor’ is defined as a tool used to hold down the tongue to allow for clear imaging of the oral and throat regions. The tongue depressor described herein may be of various shapes and sizes and may include additional features such as reflective surfaces or coatings to improve illumination within the oral cavity. The tongue depressor may be detachably secured to the supporting body, allowing for easy replacement or adjustment as necessary.

An ‘adhesive material’ as mentioned herein refers to any substance or compound capable of binding the supporting body of the invention to the surface of an image capture device or its protective case. This may include, but is not limited to, pressure-sensitive adhesives, suction-based mechanisms, or mechanical fasteners. The choice of adhesive material will depend on the specific requirements for attachment strength, durability, and ease of removal.

The term ‘light-transmitting properties’ signify the ability of certain components of the support device, such as the supporting body or an optical cover, to allow light to pass through them. This characteristic is critical for directing light from the image capture device's source towards the oral cavity, thereby enhancing the visibility and quality of captured images. Materials with such properties may include transparent or translucent plastics, glass, or any other suitable light-conducting material.

### DESCRIPTION OF DRAWINGS

The present invention relates to a support device designed to facilitate the imaging of the oral and throat regions by effectively positioning a tongue depressor relative to an image capture device. The invention addresses the challenges associated with clear and detailed imaging of these areas, which are essential for various medical diagnostics and treatments. The primary components of the invention include a tongue depressor and a supporting body, which together provide a simple yet effective solution for enhancing visibility within the oral cavity during imaging procedures.

The tongue depressor, characterized by its flat body with a rounded distal end and an opposite proximal end, is designed to hold down the tongue, thus removing one of the main obstacles to clear imaging of the throat area. The supporting body, equipped with a rear surface for detachable coupling to an image capture device, and an opposing front surface for secure attachment to the tongue depressor, ensures that the depressor is maintained in an optimal orientation perpendicular to the image plane of the image capture device. This arrangement not only improves the quality of the images obtained but also simplifies the process for medical professionals and patients alike.

This invention is adaptable to a wide range of image capture devices, including but not limited to digital cameras, smartphones, and tablets, making it a versatile tool in both clinical settings and remote consultations. The design considerations taken into account in the development of this support device ensure ease of use, reliability, and effectiveness in capturing high-quality images of the oral and throat regions.

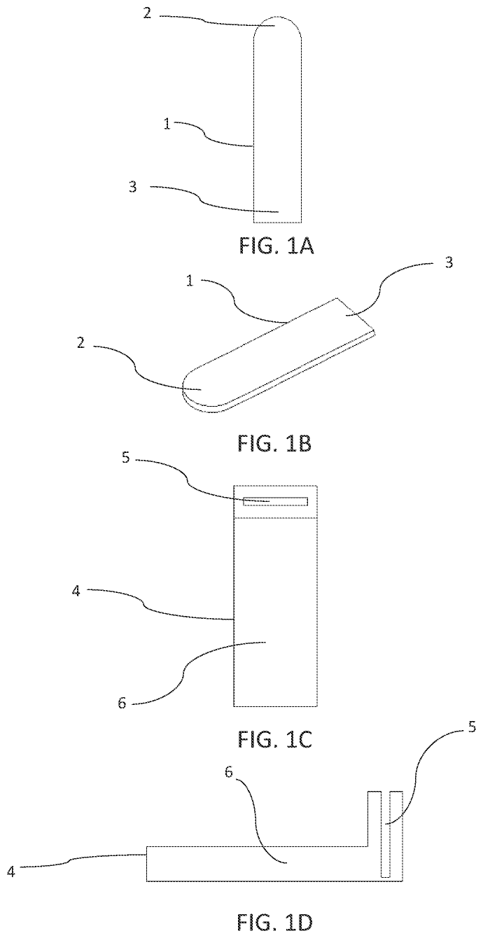

FIGS. 1A and 1B illustrate a tongue depressor (1) designed with two distinct sections: a front part (2) and a back part (3). The front part (2) is crafted to be either ellipsoidal or round, ensuring it is suitable for comfortable insertion into the mouth and throat without causing discomfort or injury, thanks to its rounded and smooth edges. The tongue depressor (1) exhibits versatility in its design, allowing for variations in symmetry, flatness, and the dimensions of the front (2), middle, and back parts (3), catering to different anatomical needs. The back part (3), which interfaces with the holding body (4), is engineered to accommodate secure attachment through features like flat, round, or sharp edges along with clip-like protrusions and indentations. This design flexibility extends to the material choices for the tongue depressor (1), which include wood, glass, silicone, metal, and plastic, among others, to match various application requirements and user preferences.

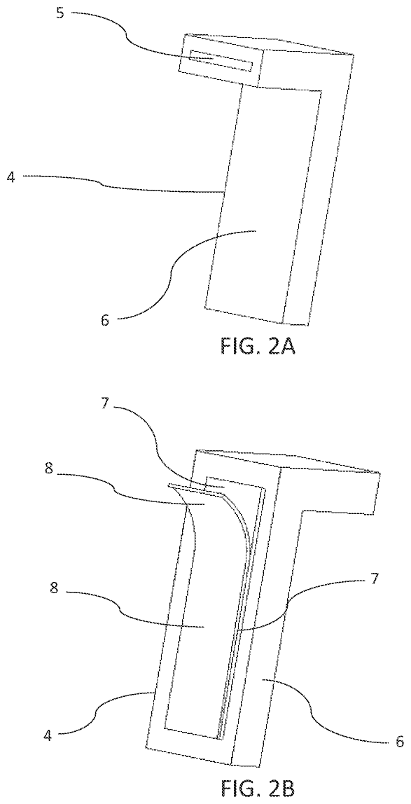

The holding body (4), depicted in FIGS. 1C, 1D, and 2A, comprises a recessed area (5) specifically designed to receive the tongue depressor (1). This area (5) is intentionally recessed to ensure a snug fit, enhancing the stability of the tongue depressor (1) when mounted. The integration of clip-like protrusions and indentations within this area (5) facilitates a secure and adjustable connection to the tongue depressor (1), accommodating different sizes and allowing for varied angular orientations relative to the handle part (6). This adjustability is crucial for achieving optimal imaging angles. The handle part (6) serves as the primary mechanism for attaching the entire assembly to an electronic device (12), ensuring the support device can be used with a broad spectrum of imaging equipment. The modular design of the recessed area (5) and the handle part (6), which can be unified into a single component or maintained as distinct entities, offers flexibility in assembly and attachment methods. Moreover, the handle part (6) is designed to accommodate varying degrees of rigidity or flexibility, thereby expanding the device's compatibility and ease of use with different electronic devices (12).

FIG. 2B illustrates the holder body (4) equipped with an adhesive surface (7) on its rear. This adhesive surface (7) is designed to facilitate temporary attachment to an electronic device (12), ensuring stability and ease of use during imaging procedures. The adhesive surface (7) is initially covered by a non-adhesive layer (8), which, when removed, activates the adhesive properties allowing for immediate use. The design permits the adhesive surface (7) to be segmented into multiple pieces or utilized as a single unit, catering to the varying sizes and configurations of electronic devices (12). This feature ensures that the holder body (4) can be detached from the handle (6) and the electronic device (12) without leaving residue, maintaining the integrity of the device.

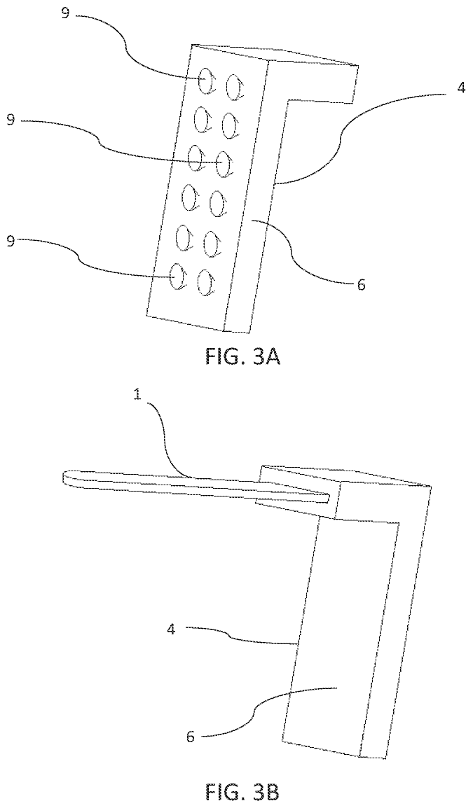

In FIG. 3A, an alternative attachment mechanism is depicted, consisting of silicone mechanical holders or suction cups (9) located on the back of the holder body (4). These suction cups (9) offer a flexible method for securing the holder body (4), and consequently the tongue depressor (1), to the electronic device (12). The number of suction cups (9) can vary, allowing for customizable attachment configurations to accommodate different electronic devices (12) effectively.

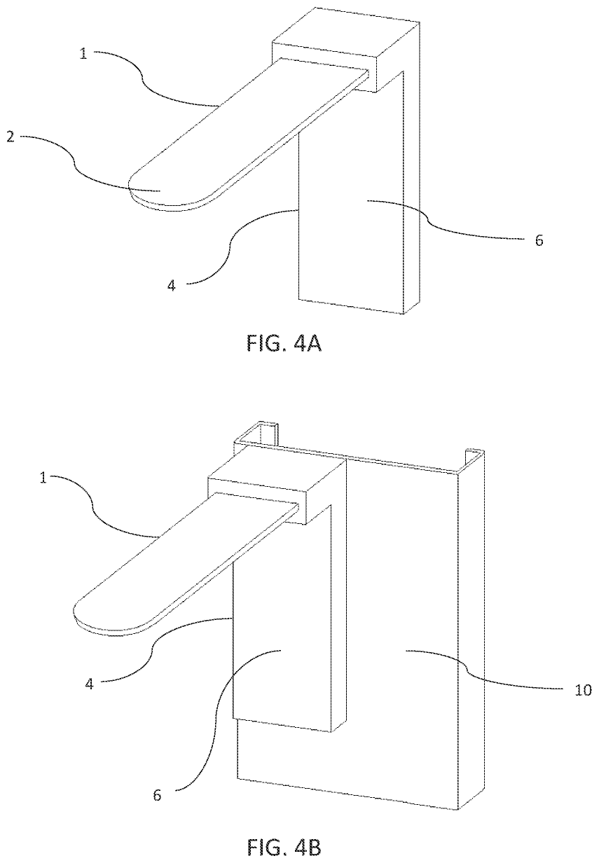

FIGS. 3B and 4A show the integration of the tongue depressor (1) with the holder body (4), forming a unified assembly. The back part (3) of the tongue depressor (1) is precisely aligned and inserted into the recess (5) of the holder body (4), ensuring that both components function as a single cohesive unit. This arrangement allows for seamless operation and enhances the device's functionality by maintaining the correct orientation and position of the tongue depressor (1) relative to the holder body (4).

FIG. 4B further demonstrates the versatility of the device assembly, showcasing the attachment of the tongue depressor (1) and the holder body (4) to a case (10) that envelops the electronic device (12). This configuration enables the holder body (4) to securely connect to the electronic device (12) indirectly via the case (10), eliminating direct contact and potential damage. Moreover, the adaptability of this setup allows the tongue depressor (1) and holder body (4) to be positioned at various locations on the case (10), accommodating different imaging requirements and electronic device (12) configurations, thereby enhancing the overall utility and flexibility of the invention.

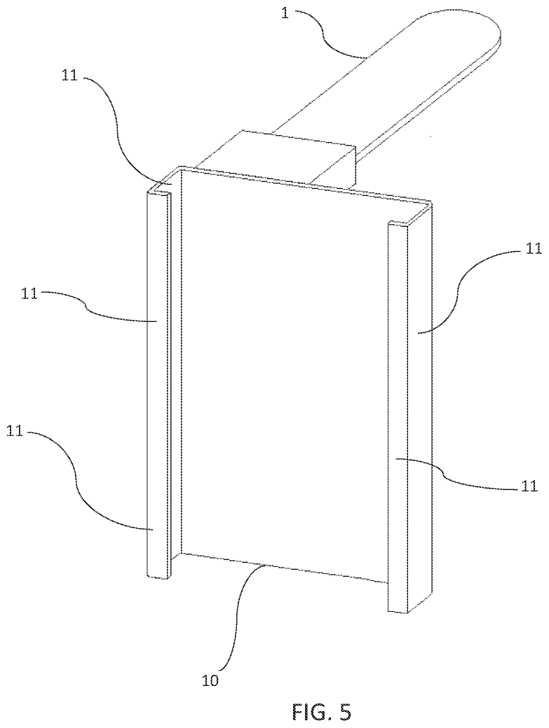

FIG. 5 showcases the case (10) equipped with edges (11) designed to engage securely with the electronic device (12). The material composition of the case (10), which may include silicone, paper, plastic, metal, or a composite of these materials, is selected for its elasticity and flexibility. This design choice ensures that the case (10) can adapt to the contours and dimensions of various electronic devices (12), thereby broadening the compatibility of the support device. The case (10) can be constructed as either a single, cohesive unit or as an assembly of multiple components, enhancing its adaptability and ease of use across different device models.

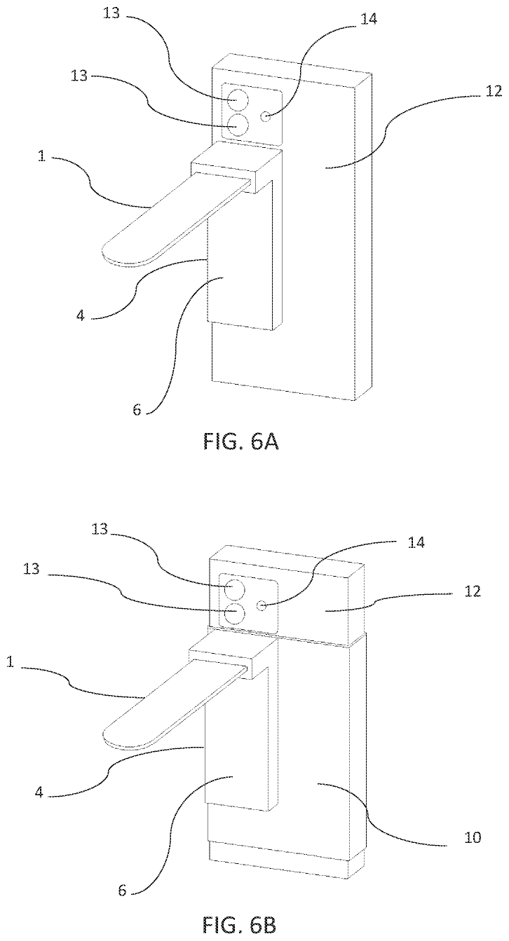

In FIG. 6A, the assembly comprising the tongue depressor (1) and the holder body (4) is positioned on the electronic device (12), strategically placed near the camera (13) and light source (14). This placement is critical to facilitate optimal imaging, as the positions of the camera (13) and light source (14) vary across electronic devices (12). The tongue depressor (1), by exerting downward pressure on the tongue, clears the visual pathway for the camera (13), enabling it to capture detailed images without obstruction.

FIG. 6B illustrates the combined setup of the tongue depressor (1), holder body (4), and case (10) on the electronic device (12). The case (10) is designed with an elastic framework to accommodate electronic devices (12) of differing sizes, ensuring a snug and secure fit. This feature allows the assembly of the tongue depressor (1) and holder body (4) to be adjusted or reconfigured based on the specific locations of the camera (13) and light source (14) on the electronic device (12). Such flexibility in the device assembly ensures that the invention can be utilized effectively with a wide range of electronic devices, optimizing the imaging process across various models and configurations.

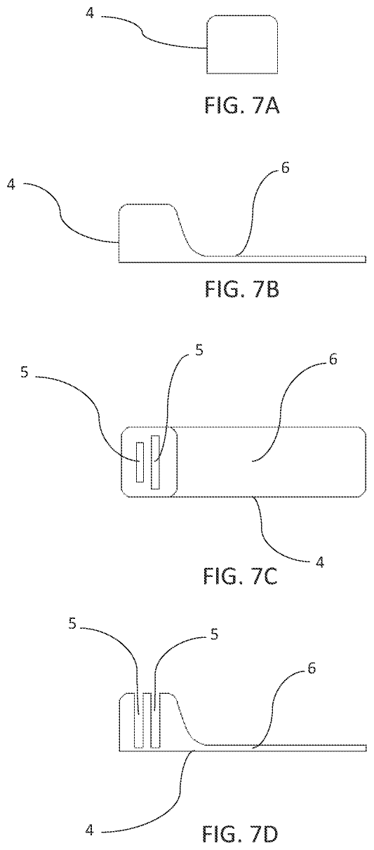

FIGS. 7A, 7B, 7C, and 7D introduce an alternative configuration of the holder body (4), which is engineered to accommodate tongue depressors (1) of various dimensions. This version of the holder body (4) features multiple recessed areas (5) for placing the tongue depressor (1), each with distinct depth and width measurements to ensure compatibility with tongue depressors (1) of different sizes. This design flexibility allows for the customization of the device to suit the specific needs of the user and enhances the utility of the holder body (4) across a broader range of applications.

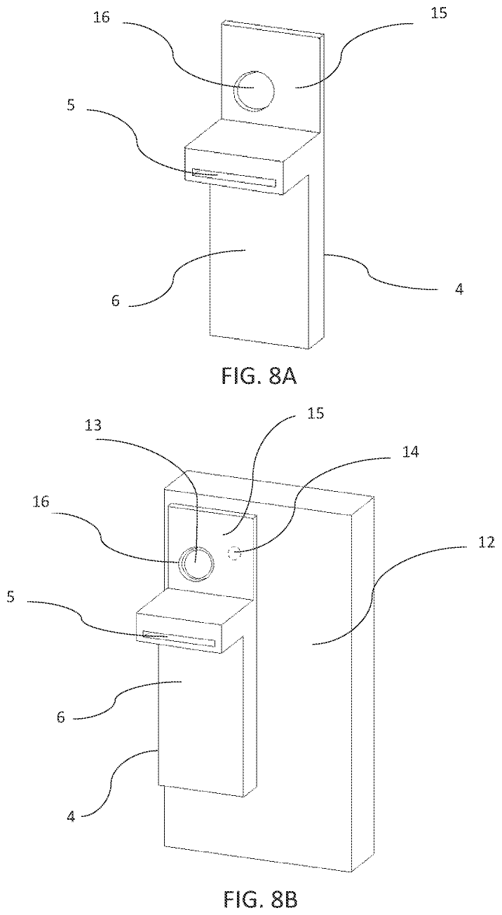

FIG. 8A details a novel aspect of the holder body (4), incorporating a transparent optical cover (15) designed to enhance light transmission to the imaging area. This optical cover (15) extends over the camera (13) and light source (14), featuring an opening (16) precisely positioned to avoid obstructing the camera's (13) field of view. The innovative use of the optical cover (15) facilitates the efficient direction of light from the source (14) towards the area of interest, leveraging fiber-optic-like properties to intensify illumination without overwhelming the subject. A selective reflective layer applied to portions of the holder body (4) further optimizes light distribution, ensuring focused illumination on the area beneath the tongue depressor (1). Variability in the dimensions of the holder body (4) and the optical cover (15) supports adaptability to different electronic devices (12) and imaging requirements.

In FIG. 8B, the integration of the holder body (4) with an electronic device (12) is illustrated, showing the alignment of the camera opening (16) with the camera (13) and the positioning of the optical cover (15) over the light source (14). The strategic design of the optical cover (15) allows for the transparent transmission of light, enhancing the illumination of the target area while minimizing glare and shadowing. The holder body's (4) structure, potentially featuring both fully transparent sections and areas with reflective or opaque treatments, plays a critical role in managing light intensity and focus, thereby improving the quality and clarity of the captured images. This approach ensures that the device can be effectively used with a wide array of electronic devices (12), offering versatility and improved functionality in oral and throat imaging applications.

The tongue depressor, designed with user safety in mind, features edges that are meticulously smoothed to ensure they do not cause harm to the tissues within the mouth. Constructed from a diverse array of materials including wood, plastic, silicone, metal, glass, or a mixture thereof, the depressor is engineered to be either tasteless and odorless or imbued with a variety of flavors or scents, such as bubblegum, strawberry, mint, cherry, and grape, catering to user preferences. Furthermore, for models of the tongue depressor that are transparent, specific sections may be enhanced with a reflective layer designed to optimize light reflection, improving visibility within the oral cavity.

The components of the throat scope (provisional name for the device), including the tongue depressor, holder body, and optical cover, may be constructed from materials that allow light to pass through, enhancing the illumination of the oral and throat regions during use. This characteristic, coupled with the option of integrating light-reflecting layers, significantly improves the device's functionality in low-light conditions. The manufacturing process of these parts involves molding or cutting techniques to achieve the desired shapes and functions, with materials chosen for their light-transmitting properties, ranging from fully transparent to opaque.

To augment the device's effectiveness, artificial intelligence (AI) models are integrated to refine stability, lighting, distance, and zoom and cropping settings, thereby elevating the quality of the images captured. These AI enhancements include classifiers for assessing image quality, deep learning algorithms for guiding users to the optimal device positioning, and advanced image fusion models at the pixel level to generate the highest quality final images. The described features and functionalities can be implemented individually or in combination, depending on the specific requirements of the application, demonstrating the invention's adaptability and comprehensive approach to improving oral and throat imaging techniques.

The entirety or each component comprising the tongue depressor (1) can be produced from biodegradable, compostable, or recyclable materials. Biodegradable plastics encompass polylactic acid (PLA), polyhydroxyalkanoates (PHA), polybutylene adipate terephthalate (PBAT), polycaprolactone (PCL), starch-based plastics, and bioplastic PET (Bio-PET). These materials, which are not limited to those mentioned, include the following characteristics: These materials, derived from renewable resources or modified from traditional plastics, offer environmentally friendly alternatives that naturally break down over time, reducing pollution and waste accumulation. Compostable materials may originate from organic matter, cellulose, or paper, while recyclable materials may include paper, glass, or metal. The stethoscope can also be produced as a combination of various materials. Production methods may include one or more of the following steps: injection molding, cutting, assembling, welding.

The production can be carried out using the following methods: The continuous flow biodegradable or recyclable plastic production method comprises several interconnected steps. Initially, biodegradable polymer pellets or granules are fed into a mixing chamber where they are combined with additives and modifiers to achieve desired material properties. The mixture is then conveyed into an extrusion unit equipped with a twin-screw extruder. Within the extruder, the polymer blend is heated, melted, and homogenized under controlled temperature and pressure conditions. Following extrusion, the molten polymer is directed into a shaping die assembly designed to impart specific geometries to the plastic products. The die assembly may include interchangeable molds or nozzles to facilitate the production of various shapes and sizes. As the plastic material exits the die, it undergoes rapid cooling and solidification, preserving the desired form. To enhance the biodegradability of the final products, the manufacturing process can incorporate additives such as bio-based fillers, enzymes, or microbial agents that accelerate decomposition in natural environments. These additives are carefully integrated into the polymer blend during mixing to ensure uniform distribution and effectiveness. Additionally, the continuous flow nature of the production line enables high throughput and efficiency, minimizing downtime and waste. Automated monitoring and control systems oversee key process parameters, ensuring consistent product quality and performance. The disclosed continuous flow biodegradable plastic production method offers a sustainable approach to plastic manufacturing, utilizing renewable resources and environmentally friendly practices to produce biodegradable plastics suitable for a wide range of applications.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the tongue depressor and support device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A support device for facilitating imaging of the oral and throat regions, the device comprising:
a tongue depressor having a flat body with a rounded distal end and an opposite proximal end; and
a supporting body, wherein the supporting body comprises:
a rear surface configured to detachably couple to an image capture device, and
an opposing front surface configured to detachably secure to the proximal end of the tongue depressor;
wherein the tongue depressor is supported at an orientation perpendicular to the image plane of the image capture device when installed in the support device.

2. The support device of claim 1, wherein the tongue depressor comprises a material selected from the group consisting of wood, plastic, silicone, metal, glass, biodegradable materials, and combinations thereof.

3. The support device of claim 1, wherein the rear surface of the supporting body includes an adhesive material covered by a protective layer, which upon removal, exposes the adhesive surface to facilitate attachment to an image capture device.

4. The support device of claim 1, wherein the supporting body further includes suction cups on its rear surface for detachable coupling to the image capture device.

5. The support device of claim 1, wherein the tongue depressor and supporting body are configured to attach to a case that is in turn configured to attach to the image capture device, facilitating compatibility with various models and sizes of mobile electronic devices.

6. The support device of claim 1, wherein the tongue depressor includes a reflective layer on at least a portion of an upper flat surface of the flat body to enhance illumination within the oral cavity.

7. The support device of claim 1, wherein the supporting body is made from a material that is at least partially transparent, capable of conducting light from the light source of the image capture device to the oral cavity.

8. The support device of claim 7, wherein the supporting body further includes a light-reflecting layer to direct light towards the oral and throat regions.

9. The support device of claim 1, wherein the front surface of the supporting body couples to the proximal end of the tongue depressor via a recessed area having a set of grooves to securely hold the tongue depressor.

10. The support device of claim 9, wherein the recessed area where the tongue depressor is placed is configured to allow for different orientations of the tongue depressor relative to the supporting body, enabling adjustable angles for optimal imaging.

11. The support device of claim 1, further comprising an optical cover with light-transmitting properties connected to the supporting body, designed to align with the camera and light source of the image capture device for improved image capture.

12. The support device of claim 11, wherein the optical cover includes an opening for the camera that does not interfere with the camera's field of view and is configured to direct light from the light source towards the oral cavity.

13. The support device of claim 1, wherein the device utilizes artificial intelligence models to optimize image capture parameters including stability, lighting, distance, and zoom and cropping settings, thereby enhancing the quality of images collected.

14. The support device of claim 13, wherein the artificial intelligence models include classifiers for image-quality assessment and deep learning models for assisting in the optimal positioning of the device and for pixel-level image fusion to produce the final image.

15. A continuous flow biodegradable plastic production method comprising:
mixing biodegradable polymer materials with additives and modifiers in a mixing chamber;
extruding the polymer blend using a twin-screw extruder under controlled temperature and pressure conditions;
shaping the molten polymer into desired products using a shaping die assembly;
incorporating biodegradability-enhancing additives into the polymer blend during mixing to accelerate decomposition in natural environments;
automating monitoring and control systems to ensure consistent product quality and performance.
